(19) United States
(12) Patent Application Publication
(10) Pub. No.: US 2025/0296167 A1
(43) Pub. Date: Sep. 25, 2025

(54) ENHANCED DEVICES, SYSTEMS, AND METHODS FOR MANUFACTURING UNDERCUT THREADFORMS ON FASTENERS

(71) Applicant: RTG Scientific, LLC, Austin, TX (US)

(72) Inventors: Harrison Cole HOLDEN, Logan, UT (US); Rulon Jacob BAUER, Preston, ID (US)

(21) Appl. No.: 19/088,618

(22) Filed: Mar. 24, 2025

Related U.S. Application Data
(60) Provisional application No. 63/569,174, filed on Mar. 24, 2024.

(51) Int. Cl. B23G 5/18; A61B 17/86; B23G 1/32

(52) U.S. Cl. CPC B23G 5/18; A61B 17/866; B23G 1/32; B23G 2200/48

(57) ABSTRACT

Opposing undercut surfaces may be formed on a helical thread disposed about a shaft of a fastener by: rotating the shaft; translating a first cutting head medially toward the shaft along a first oblique trajectory of a first oblique cutting surface of the first cutting head; translating the first cutting head along a length of the shaft to form at least one of the opposing undercut surfaces on the helical thread; translating a second cutting head medially toward the shaft along a second oblique trajectory of a second oblique cutting surface of the second cutting head; and translating the second cutting head along a length of the shaft to form at least another one of the opposing undercut surfaces on the helical thread.

### CROSS-REFERENCE TO RELATED APPLICATIONS

This application claims the benefit of U.S. Provisional Patent Application Ser. No. 63/569,174 filed on Mar. 24, 2024, entitled “ENHANCED SYSTEMS AND METHODS FOR MANUFACTURING UNDERCUT FASTENER THREADFORMS”. The above-referenced document is hereby incorporated by reference in its entirety.

### TECHNICAL FIELD

The present disclosure relates to enhanced devices, systems, and methods for manufacturing fasteners. More specifically, the present disclosure relates to enhanced devices, systems, and methods for manufacturing undercut threadforms on fasteners.

### BACKGROUND

Surgical procedures involving fasteners implanted within bone and other tissues can become lose over time due to multi-axial forces and off-axis loading scenarios that may be applied to the fastener during the healing process. Traditional fastener thread designs may not provide sufficient fastener fixation to overcome these multi-axial forces and off-axis loading scenarios.

Improved fastener thread designs that utilize undercut threadforms can increase bone fixation and load sharing between a bone/fastener interface experiencing multi-axial and off-loading conditions. However, these improved undercut threadform shapes can be difficult to manufacture. Accordingly, enhanced devices, systems, and methods for manufacturing undercut threadforms on fasteners would be desirable.

### SUMMARY

The enhanced devices, systems, and methods of the present disclosure have been developed in response to the present state of the art, and in particular, in response to the problems and needs in the art that have not yet been fully solved by currently available devices, systems, and methods for manufacturing undercut threadforms on fasteners.

In some embodiments, a method of manufacturing a fastener may include forming opposing undercut surfaces of a helical thread disposed about a shaft of the fastener via a first cutting process and a second cutting process. The first cutting process may include: (1) placing a first cutting head of a first mill tool at a first lateral position relative to the shaft; (2) rotating the shaft about a longitudinal axis of the shaft; (3) translating the first cutting head medially toward a first medial position relative to the shaft along a first oblique trajectory defined by a first oblique cutting surface of the first cutting head; and (4) translating the first cutting head along at least a first portion of a length of the shaft to form at least one of the opposing undercut surfaces on the helical thread. The second cutting process may include: (1) placing a second cutting head of a second mill tool at a second lateral position relative to the shaft; (2) rotating the shaft about the longitudinal axis of the shaft; (3) translating the second cutting head medially toward a second medial position relative to the shaft along a second oblique trajectory defined by a second oblique cutting surface of the second cutting head; and (4) translating the second cutting head along at least a second portion of the length of the shaft to form at least another one of the opposing undercut surfaces on the helical thread.

The method according to any preceding paragraph, wherein the first medial position may include a plurality of first medial positions along the first oblique trajectory, and the second medial position may include a plurality of second medial positions along the second oblique trajectory.

The method according to any preceding paragraph, wherein the first cutting head may be shaped to form a third undercut surface and a fifth open surface of the helical thread, and the second cutting head may be shaped to form a first undercut surface, a second undercut surface, and a fourth open surface of the helical thread.

The method according to any preceding paragraph, wherein the first cutting head may be shaped to form a third undercut surface and a fourth open surface of the helical thread, and the second cutting head may be shaped to form a first undercut surface, a second undercut surface, and a fifth open surface of the helical thread.

The method according to any preceding paragraph, wherein the first cutting head may be placed on a first side of the shaft at the first lateral position, and the second cutting head may be placed on a second side of the shaft at the second lateral position. The first cutting head and the second cutting head may be separated by a selected degree of rotation about the longitudinal axis of the shaft.

The method according to any preceding paragraph, wherein the first cutting head and the second cutting head may be separated by 180 degrees of rotation about the longitudinal axis of the shaft.

The fastener formed by the process according to any preceding paragraph, wherein the fastener may include a tapered shape, and the first cutting head and the second cutting head may be translated along the tapered shape of the fastener to form the opposing undercut surfaces on the helical thread.

In some embodiments, a method of manufacturing a fastener may include forming at least two opposing undercut surfaces of at least one helical thread disposed about a shaft of the fastener via a cutting process by: (1) placing a cutting head of a mill tool at a first position along the shaft; (2) rotating the shaft about a longitudinal axis of the shaft; and (3) translating the cutting head along at least a portion of a length of the shaft to form the at least two opposing undercut surfaces on the at least one helical thread.

The method according to any preceding paragraph, wherein the cutting head does not rotate about a longitudinal axis of the mill tool during the cutting process that forms the at least two opposing undercut surfaces.

The method according to any preceding paragraph, wherein the fastener includes a tapered shape, and the cutting head is translated along the tapered shape of the fastener to form the at least two opposing undercut surfaces on the at least one helical thread.

The method according to any preceding paragraph, wherein the at least one helical thread may include a first helical thread disposed about the shaft of the fastener, the cutting head may be shaped to simultaneously form the at least two opposing undercut surfaces on the first helical thread as the cutting head is translated along the shaft, and the at least two opposing undercut surfaces may include a third undercut surface and at least one of a first undercut surface and a second undercut surface on the first helical thread.

The method according to any preceding paragraph, wherein the at least two opposing undercut surfaces may include the first undercut surface, the second undercut surface, and the third undercut surface on the first helical thread.

The method according to any preceding paragraph, wherein the at least one helical thread may include a first helical thread disposed about the shaft of the fastener, and a second helical thread disposed about the shaft of the fastener adjacent the first helical thread. The cutting head may be shaped to simultaneously form the at least two opposing undercut surfaces as the cutting head is translated along the shaft, and the at least two opposing undercut surfaces may include a third undercut surface on the first helical thread and a seventh undercut surface on the second helical thread.

The method according to any preceding paragraph, wherein the at least one helical thread may include a first helical thread disposed about the shaft of the fastener, and a second helical thread disposed about the shaft of the fastener adjacent the first helical thread. The cutting head may be shaped to simultaneously form the at least two opposing undercut surfaces as the cutting head is translated along the shaft, and the at least two opposing undercut surfaces may include at least one of a first undercut surface on the first helical thread and a second undercut surface on the first helical thread, as well as at least one of a fifth undercut surface on the second helical thread and a sixth undercut surface on the second helical thread.

The fastener formed by the process according to any preceding paragraph, wherein the at least two opposing undercut surfaces may include the first undercut surface and the second undercut surface on the first helical thread, as well as the fifth undercut surface and the sixth undercut surface on the second helical thread.

In some embodiments, a method of manufacturing a fastener may include forming opposing undercut surfaces of a helical thread disposed about a shaft of the fastener via a first cutting process, a second cutting process, and a third cutting process. The first cutting process may include: placing a first cutting head of a first mill tool at a first position along the shaft; rotating the shaft in a first rotational direction about a longitudinal axis of the shaft; and translating the first cutting head along at least a first portion of a length of the shaft to form a fourth open surface and a fifth open surface of the helical thread. The second cutting process may include placing a second cutting head of a second mill tool at a second position along the shaft; rotating the shaft in the first rotational direction about the longitudinal axis of the shaft; and translating the second cutting head along at least a second portion of the length of the shaft to form a first undercut surface and a second undercut surface of the helical thread. The third cutting process may include: placing a third cutting head of a third mill tool at a third position along the shaft; rotating the shaft in the first rotational direction about the longitudinal axis of the shaft; and translating the third cutting head along at least a third portion of the length of the shaft to form a third undercut surface of the helical thread.

The method according to any preceding paragraph, wherein two or more of: the first cutting process, the second cutting process, and the third cutting process may be performed simultaneously.

The method according to any preceding paragraph, wherein at least two of: the first cutting head, the second cutting head, and the third cutting head may be placed on opposing sides of the shaft with respect to each other to balance two or more cutting forces applied to the shaft during the cutting processes.

The method according to any preceding paragraph, wherein the first cutting process, the second cutting process, and the third cutting process may be performed individually, and in any sequence.

The method according to any preceding paragraph, wherein the fastener may include a tapered shape.

These and other features and advantages of the present disclosure will become more fully apparent from the following description and appended claims or may be learned by the practice of the enhanced devices, systems, and methods for manufacturing undercut threadforms on fasteners set forth hereinafter.

### BRIEF DESCRIPTION OF THE DRAWINGS

Exemplary embodiments of the present disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the present disclosure will be described with additional specificity and detail through use of the accompanying drawings in which:

FIG. 1A illustrates a perspective distal end view of a fastener, according to an embodiment of the present disclosure;

FIG. 1B illustrates a perspective proximal end view of the fastener of FIG. 1A;

FIG. 1C illustrates a side view of the fastener of FIG. 1A;

FIG. 1D illustrates a cross-sectional side view of the fastener taken along the line A-A in FIG. 1C;

FIG. 2 illustrates a partial cross-sectional side view of a fastener comprising crescent-shaped threading;

FIG. 3A illustrates a perspective distal end view of a fastener, according to another embodiment of the present disclosure;

FIG. 3B illustrates a perspective proximal end view of the fastener of FIG. 3A;

FIG. 3C illustrates a side view of the fastener of FIG. 3A;

FIG. 3D illustrates a cross-sectional side view of the fastener of FIG. 3A taken along the line B-B shown in FIG. 3C;

FIG. 4A illustrates a perspective distal end view of a fastener, according to another embodiment of the present disclosure;

FIG. 4B illustrates a perspective proximal end view of the fastener of FIG. 4A;

FIG. 4C illustrates a side view of the fastener of FIG. 4A;

FIG. 4D illustrates a cross-sectional side view of the fastener of FIG. 4A taken along the line C-C shown in FIG. 4C;

FIG. 5 illustrates a partial cross-sectional side view of a fastener undergoing various steps of a cutting process, according to an embodiment of the present disclosure;

FIG. 6A illustrates a partial cross-sectional side view of a fastener undergoing a first step of a cutting process, according to another embodiment of the present disclosure;

FIG. 6B illustrates the fastener of FIG. 6A undergoing a second step of the cutting process;

FIG. 7A illustrates a partial cross-sectional side view of a fastener undergoing a first step of a cutting process, according to another embodiment of the present disclosure;

FIG. 7B illustrates the fastener of FIG. 7A undergoing a second step of the cutting process;

FIG. 8 illustrates a partial cross-sectional side view of a fastener undergoing the cutting steps of FIGS. 7A and 7B on opposing sides of the fastener, according to an embodiment of the present disclosure;

FIG. 9 illustrates a partial cross-sectional side view of a fastener undergoing a step of a cutting process, according to another embodiment of the present disclosure;

FIG. 10A illustrates a perspective view of a fastener undergoing a step of a cutting process, according to another embodiment of the present disclosure;

FIG. 10B illustrates a side view of the fastener shown in FIG. 10A;

FIG. 10C illustrates a cross-sectional side view of the fastener shown in FIG. 10B;

FIG. 11A illustrates a perspective view of a fastener undergoing a step of a cutting process, according to another embodiment of the present disclosure;

FIG. 11B illustrates a side view of the fastener shown in FIG. 11A;

FIG. 11C illustrates a cross-sectional side view of the fastener shown in FIG. 11B;

FIG. 12A illustrates a perspective distal end view of a tapered fastener, according to another embodiment of the present disclosure;

FIG. 12B illustrates a perspective proximal end view of the tapered fastener of FIG. 12A;

FIG. 12C illustrates a side view of the tapered fastener of FIG. 12A;

FIG. 12D illustrates a cross-sectional side view of the tapered fastener of FIG. 12A taken along the line D-D shown in FIG. 12C; and

FIG. 13 illustrates a partial cross-sectional side view of a tapered fastener undergoing a cutting process, according to another embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms may also be applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means outboard deviation of the knees (away from the sagittal plane) from the line between the hip and ankle, resulting in a “bowlegged” stance. Valgus means inboard deviation of the knees (toward the sagittal plane) from the line between the hip and ankle, resulting in a “knock-kneed” stance.

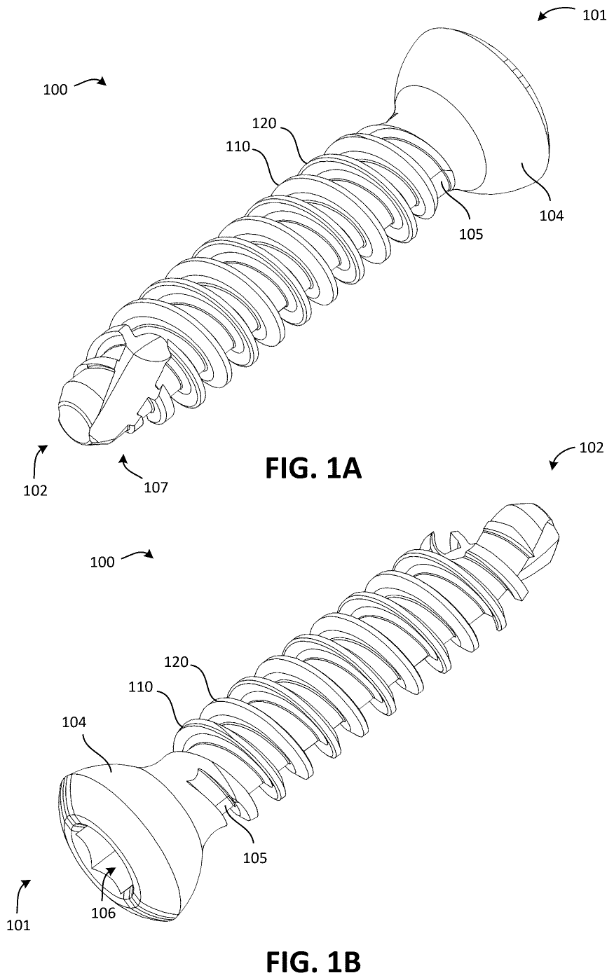

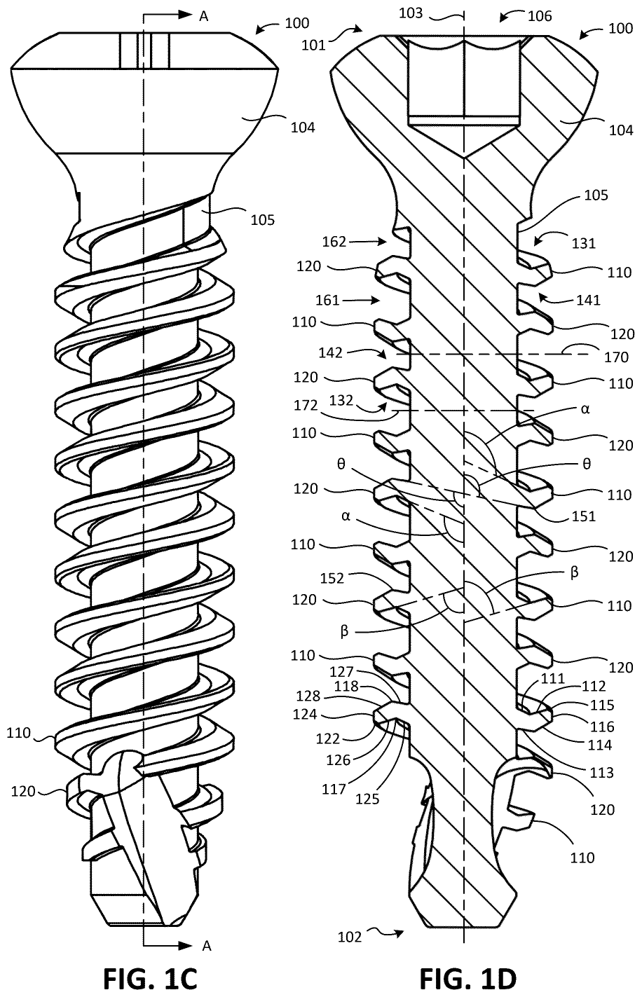

FIGS. 1A-D illustrate various views of a device, screw, bone screw, implant, or fastener 100, according to an example of the present disclosure. Specifically, FIG. 1A is a perspective distal end view of the fastener, FIG. 1B is a perspective proximal end view of the fastener, FIG. 1C is a side view of the fastener, and FIG. 1D is a cross-sectional side view of the fastener taken along the line A-A in FIG. 1C.

In general, the fastener may include a shaft 105 having a proximal end 101, a distal end 102, and a longitudinal axis 103. The fastener may also include a head 104 located at the proximal end 101 of the shaft 105, a torque connection interface 106 formed in/on the head 104 (in either a male or female configuration), and a self-tapping feature 107 formed in the shaft 105, such as the distal end 102 of the shaft 105, etc.

In some embodiments, the fastener may include a first helical thread 110 disposed about the shaft 105, and/or a second helical thread 120 disposed about the shaft 105 adjacent the first helical thread 110.

In some embodiments, the fastener may include a “dual start” or “dual lead” thread configuration comprising the first helical thread 110 and the second helical thread 120.

In some embodiments, a depth of the first helical thread 110 and/or the second helical thread 120 with respect to the shaft 105 may define a major diameter vs. a minor diameter of the shaft 105 alone.

In some embodiments, a major diameter and/or a minor diameter of the fastener may be constant or substantially constant along the entire length of the fastener, along a majority of the length of the fastener, or along any length of the fastener. In these embodiments, a constant minor diameter may help avoid blowout of narrow/delicate bones (e.g., a pedicle or other bones) when inserting a fastener into a bone. In some embodiments, a pilot hole may first be drilled into a narrow/delicate bone and then a fastener having a similar minor diameter in comparison to the diameter of the pilot hole may be chosen to avoid blowout when inserting the fastener into the bone.

In some embodiments, a depth of the first helical thread 110 and/or the second helical thread 120 with respect to the shaft 105 may vary along a length of the shaft 105 to define one or more major diameters of the fastener and/or one or more regions along the fastener may comprise a one or more continuously variable major diameters.

In some embodiments, a thickness of the shaft 105 may vary along a length of the shaft 105 to define one or more minor diameters of the fastener, and/or one or more regions along the fastener may comprise one or more continuously variable minor diameters. In some embodiments, a thickness/height/width/length/pitch/angle/shape, etc., of the first helical thread 110 and/or the second helical thread 120 (or any additional helical thread) may vary along a length of the shaft 105. For example, a thickness/height/width/length/pitch/angle/shape, etc., of the first helical thread 110 and/or the second helical thread 120 may be greater towards the tip of the fastener and thinner towards the head of the fastener (or vice versa) in either a discrete or continuously variable fashion, etc., or combinations thereof.

In some embodiments, the major and/or minor diameters may increase toward a proximal end or head of a fastener (or vice versa) in order to increase bone compaction as the fastener is terminally inserted into the bone/tissue.

In some embodiments, a pitch of the first helical thread 110 and/or the second helical thread 120 may vary along a length of the fastener.

In some embodiments, the fastener may include a plurality of helical threads disposed about the shaft 105. However, it will also be understood that any of the fasteners disclosed or contemplated herein may include a single helical thread disposed about the shaft of the fastener. Moreover, the fastener may comprise a nested plurality of helical threads having different lengths (not shown). As one non-limiting example, the fastener may include a first helical thread 110 that is longer than a second helical thread 120, such that the fastener comprises dual threading along a first portion of the shaft 105 and single threading along a second portion of the shaft 105.

In some embodiments, the plurality of helical threads may include three helical threads comprising a “triple start” or “triple lead” thread configuration (not shown).

In some embodiments, the plurality of helical threads may include four helical threads comprising a “quadruple start” or “quadruple lead” thread configuration (not shown).

In some embodiments, the plurality of helical threads may include more than four helical threads (not shown).

In some embodiments, the fastener may include first threading with any of the shapes disclosed herein oriented toward one of the proximal end and the distal end of the fastener, with the first threading located proximate the distal end of the fastener, as well as second threading with any of the shapes disclosed herein oriented toward the other one of the proximal end and the distal end of the fastener, with the second threading located proximate the head of the fastener (not shown).

In some embodiments, the fastener may include multiple threading (e.g., dual helical threading, etc.) with any of the shapes disclosed herein located proximate one of the proximal end and the distal end of the fastener, as well as single threading with any of the shapes disclosed herein with the second threading located proximate the other of the proximal end and the distal end of the fastener.

In some embodiments, the first helical thread 110 may include a plurality of first concave undercut surfaces 131 and a plurality of first convex undercut surfaces 141.

In some embodiments, the second helical thread 120 may include a plurality of second concave undercut surfaces 132 and a plurality of second convex undercut surfaces 142.

In some embodiments, when the fastener is viewed in section along a plane that intersects the longitudinal axis 103 of the shaft 105 (e.g., see FIG. 1D), the plurality of first concave undercut surfaces 131 and the plurality of second convex undercut surfaces 142 may be oriented toward (i.e., point toward) the proximal end 101 of the shaft 105.

In some embodiments, the plurality of first convex undercut surfaces 141 and the plurality of second concave undercut surfaces 132 may be oriented toward (i.e., point toward) the distal end 102 of the shaft 105.

In some embodiments, at least one of the plurality of first concave undercut surfaces 131, the plurality of first convex undercut surfaces 141, the plurality of second concave undercut surfaces 132, and the plurality of second convex undercut surfaces 142 may comprise at least one substantially flat surface.

In some embodiments, when the fastener is viewed in section along a plane intersecting the longitudinal axis 103 of the shaft 105, the first helical thread 110 may comprise a plurality of first bent shapes (comprising at least one surface that is angled relative to the longitudinal axis 103 of the shaft 105 and/or at least one undercut surface) with a plurality of first intermediate portions 151 that are oriented toward (i.e., point toward) the distal end 102 of the shaft 105. This may be referred to as “standard” threading, having a “standard” orientation.

In some embodiments, when the fastener is viewed in section along a plane intersecting the longitudinal axis 103 of the shaft 105, the second helical thread 120 may comprise a plurality of second bent shapes (comprising at least one surface that is angled relative to the longitudinal axis 103 of the shaft 105 and/or at least one undercut surface) with a plurality of second intermediate portions 152 that are oriented toward (i.e., point toward) the proximal end 101 of the shaft 105. This may be referred to as “inverted” threading, having an “inverted” orientation.

In some embodiments, one or more helical threads may morph/transition between a standard orientation and an inverted orientation along a shaft of a fastener.

In some embodiments, at least one of the plurality of first concave undercut surfaces 131, the plurality of first convex undercut surfaces 141, the plurality of second concave undercut surfaces 132, and the plurality of second convex undercut surfaces 142 may comprise at least one curved surface.

As shown in FIG. 1D, the proximally-oriented and distally-oriented surfaces of the first helical thread 110 (i.e., the first concave undercut surfaces 131 and the first convex undercut surfaces 141 in the fastener of FIG. 1D) may not have mirror symmetry relative to each other about any plane perpendicular to the longitudinal axis 103 of the fastener. Rather, the first concave undercut surfaces 131 and the first convex undercut surfaces 141 may be generally parallel to each other. The same may be true for the second helical thread 120, in which the second concave undercut surfaces 132 and the second convex undercut surfaces 142 may not have mirror symmetry relative to each other, but may be generally parallel to each other.

Conversely, as also shown in FIG. 1D, the proximally-oriented surfaces of the first helical thread 110 may have mirror symmetry relative to the distally-oriented surfaces of the second helical thread 120. Specifically, the first concave undercut surfaces 131 may have mirror symmetry relative to the second convex undercut surfaces 142 about a plane 170 that bisects the space between them, and lies perpendicular to the longitudinal axis 103.

Similarly, the distally-oriented surfaces of the first helical thread 110 may have mirror symmetry relative to the proximally-oriented surfaces of the second helical thread 120. Specifically, the second concave undercut surfaces 132 may have mirror symmetry relative to the first convex undercut surfaces 141 about a plane 172 that bisects the space between them, and lies perpendicular to the longitudinal axis 103.

This mirror symmetry may be present along most of the length of the first helical thread 110 and the second helical thread 120, with symmetry across different planes arranged between adjacent turns of the first helical thread 110 and the second helical thread 120 along the length of the longitudinal axis 103. Such mirror symmetry may help more effectively capture bone between the first helical thread 110 and the second helical thread 120, and may also facilitate manufacture of the fastener.

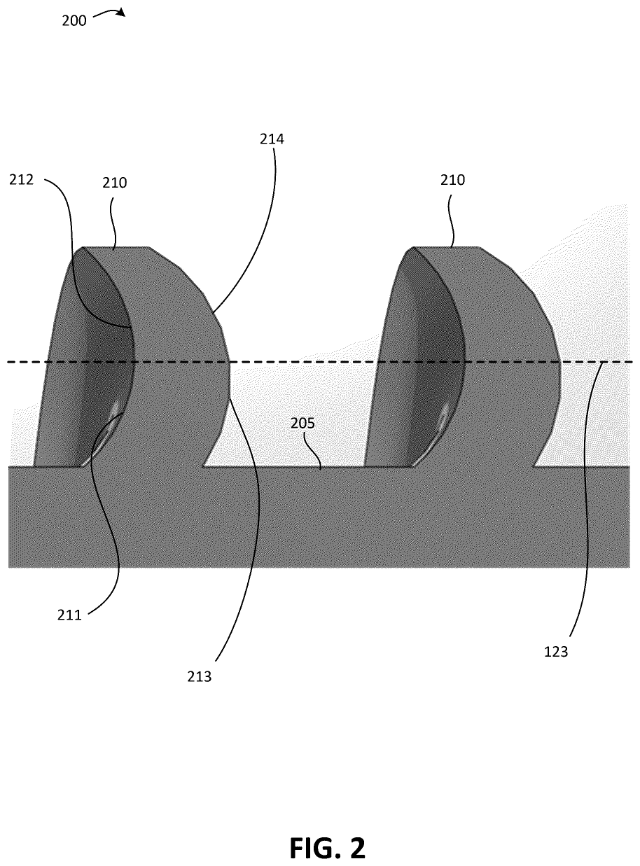

In some embodiments, when the fastener is viewed in section along a plane intersecting the longitudinal axis 103 of the shaft 105, the first helical thread 110 may include at least one partial crescent shape that is oriented toward (i.e., points toward) the distal end 102 of the shaft 105 and/or the proximal end 101 of the shaft 105. FIG. 2 illustrates a partial cross-sectional view of a fastener 200 comprising crescent shapes, as one non-limiting example of such an embodiment.

In some embodiments (not shown), when the fastener is viewed in section along a plane intersecting the longitudinal axis of the shaft, a helical thread 210 may include at least one partial crescent shape that is oriented toward (i.e., points toward) the distal end of the shaft 205, and a second helical thread may include at least one partial crescent shape that is oriented toward (i.e., points toward) the proximal end of the shaft 205.

In some embodiments (not shown), the helical thread may include a first plurality of partial crescent shapes that are oriented toward (i.e., point toward) the distal end of the shaft, and the second helical thread may include a second plurality of partial crescent shapes that are oriented toward (i.e., point toward) the proximal end of the shaft.

In some embodiments (not shown), the first plurality of partial crescent shapes and the second plurality of partial crescent shapes may be arranged in alternating succession along the shaft of the fastener.

In some embodiments, the helical thread 210 may be bisected by the line 123 shown in FIG. 2 with each crescent shape including a plurality of first crescent undercut surfaces 211, a plurality of second crescent undercut surfaces 212, a plurality of third crescent undercut surfaces 213, and a plurality of fourth crescent open surfaces 214 similar to the helical threading shown in FIG. 1D, except with curved surfaces in place of flat surfaces.

In some embodiments, the plurality of first crescent undercut surfaces 211 and the plurality of second crescent undercut surfaces 212 may comprise concave curved surfaces. However, it will be understood that portions of the plurality of first crescent undercut surfaces 211 and/or portions of the plurality of second crescent undercut surfaces 212 may also comprise convex curved surfaces and/or flat surfaces (not shown in FIG. 2).

In some embodiments, the plurality of third crescent undercut surfaces 213 and the plurality of fourth crescent open surfaces 214 may comprise convex curved surfaces. However, it will be understood that portions of the plurality of third crescent undercut surfaces 213 and the plurality of fourth crescent open surfaces 214 may also comprise concave curved surfaces and/or flat surfaces (not shown in FIG. 2).

In some embodiments, the plurality of third crescent undercut surfaces 213 and the plurality of fourth crescent open surfaces 214 may be replaced by a ramped surface (such as that utilized in a standard buttress thread design) without any undercuts (not shown in FIG. 2). Likewise, any of the other thread designs disclosed herein may utilize a ramped or buttress thread design on at least one side of the helical thread.

In some embodiments, a fastener may have only standard threads or only inverted threads. The type of threads that are desired may depend on the type and/or magnitude of loads to be applied to the fastener. For example, a fastener loaded axially away from the bone in which it is implanted may advantageously have a standard thread, while a fastener loaded axially toward the bone in which it is implanted may advantageously have an inverted thread. A fastener that may experience multi-axial loading and/or off-loading conditions may advantageously include at least one standard thread and at least one inverted thread in order to increase bone fixation and load sharing between a bone/fastener interface during multi-axial and off-loading conditions to reduce high bone strain and distribute multi-axial forces applied to the bone in a load-sharing, rather than load-bearing, configuration. Shear loads and/or bending moments may also be optimally resisted with any chosen combination of threading, threading morphology, and/or threading variations contemplated herein to optimally resist shear loads, bending moments, multi-axial loading, off-loading conditions, etc.

In some embodiments, fasteners with standard threads may be used in conjunction with fasteners with inverted threads in order to accommodate different loading patterns.

In some embodiments, a single fastener may have both standard and inverted threads, like the fastener shown in FIGS. 1A-1D. Such a combination of threads may help the fastener remain in place with unknown and/or varying loading patterns.

In some embodiments, the geometry of the threading of a fastener (with standard and/or inverted threads) may be varied to suit the fastener for a particular loading scheme. For example, the number of threads, the number of thread starts, the pitch of the threading, the lead(s) of the threading, the shape(s) of the threading, any dimension(s) associated with the threading (e.g., any length(s)/width(s)/height(s)/inflection-point(s), etc. associated with the threading), the major diameter(s), the minor diameter(s), any angulation/angles associated with any surfaces of the threading, the “handedness” of the threading (e.g., right-handed vs. left-handed), etc., may be varied accordingly to suit any specific medium of installation, loading pattern, desired radial loading force, pull-out strength, application, procedure, etc., that may be involved.

In some embodiments, the material(s) of any portion of a fastener, implant, or instrument described or contemplated herein may include, but are not limited to: metals (e.g., titanium, cobalt, stainless steel, etc.), metal alloys, plastics, polymers, ceramics, PEEK, UHMWPE, composites, additive particles, textured surfaces, biologics, biomaterials, bio-resorbable materials, bone, etc. Likewise, any fastener, implant, or instrument described or contemplated herein may be formed by any manufacturing method including, but not limited to: CNC manufacturing, injection molding, 3D printing, etc., and may include a solid or porous surface to encourage tissue/bone in-growth and/or may include any coating or surface treatment to stimulate tissue/bone growth, exhibit anti-microbial properties, etc.

In some embodiments, any of the fasteners or implants described herein may include additional features such as: self-tapping features, locking features (e.g., locking threading formed on a portion of the fastener, such as threading located on or near a head of the fastener), opening(s), longitudinal passageways (e.g., to receive a guide wire therethrough), cannulation(s), fenestration(s), any style of fastener head (or no fastener head at all), any style of torque connection interface (or no torque connection interface at all), etc.

In some embodiments, any of the fasteners or implants described herein may also include opening(s), cannulation(s), fenestration(s), etc., that may be configured to receive any suitable bone cement or bone augment material therein to facilitate bone in-growth, bone fusion, etc.

In some embodiments, a tap tool may be utilized to pre-form threading in a bone or bone augment material (or in any other substrate) according to any threading shape that is disclosed or contemplated herein. In this manner, tap tools with any suitable shape may be utilized in conjunction with any fastener described or contemplated herein to match or substantially match the threading geometry of a given fastener or implant.

In some embodiments, a minor diameter of the fastener may be selected to match, or substantially match, a diameter of a pilot hole that is formed in a bone to avoid bone blowout when the fastener is inserted into the pilot hole.

Additionally, or alternatively thereto, the type of threads and/or thread geometry may be varied based on the type of bone in which the fastener is to be anchored. For example, fasteners anchored in osteoporotic bone may fare better with standard or inverted threads, or when the pitch, major diameter, and/or minor diameter are increased or decreased, or when the angulation of thread surfaces is adjusted, etc.

In some embodiments, a surgical kit may include one or more fasteners or any other implants that utilize any of the threadforms/features described or contemplated herein, as well as one or more other devices/instruments (e.g., blades, plates, rods, pins, nails, wires, bone screws, etc.). The surgeon may select the appropriate components from the kit based on the particular loads to be applied and/or the quality of bone in which the implants(s) are to be anchored.

Continuing with FIG. 1D, in some embodiments the first helical thread 110 may include a plurality of first undercut surfaces 111, a plurality of second undercut surfaces 112, a plurality of third undercut surfaces 113, a plurality of fourth open surfaces 114, a plurality of fifth open surfaces 115, a plurality of sixth outer surfaces 116, a first inflection point 117, and a second inflection point 118.

In some embodiments, the second helical thread 120 may include a plurality of fifth undercut surfaces 125, a plurality of sixth undercut surfaces 126, a plurality of seventh undercut surfaces 127, a plurality of eighth open surfaces 128, a plurality of second open surfaces 122, and a plurality of fourth outer surfaces 124.

In some embodiments one or more of the plurality of first undercut surfaces 111, the plurality of second undercut surfaces 112, the plurality of third undercut surfaces 113, the plurality of fourth open surfaces 114, the plurality of fifth undercut surfaces 125, the plurality of sixth undercut surfaces 126, the plurality of seventh undercut surfaces 127, and the plurality of eighth open surfaces 128 may comprise at least one flat or substantially flat surface.

In some embodiments, the plurality of first undercut surfaces 111, the plurality of third undercut surfaces 113, the plurality of sixth undercut surfaces 126, and the plurality of eighth open surfaces 128 may be angled towards the distal end 102 of the shaft 105.

In some embodiments, the plurality of second undercut surfaces 112, the plurality of fourth open surfaces 114, the plurality of fifth undercut surfaces 125, and the plurality of seventh undercut surfaces 127 may be angled towards the proximal end 101 of the shaft 105.

In some embodiments, when the fastener is viewed in section along a plane that intersects the longitudinal axis 103 of the shaft 105 (as shown in FIG. 1D), the first helical thread 110 may include at least one chevron shape that is oriented toward (i.e., points toward) the distal end 102 of the shaft 105. Likewise, the second helical thread 120 may also include at least one chevron shape that is oriented toward (i.e., points toward) the proximal end 101 of the shaft 105.

In some embodiments, when the fastener is viewed in section along a plane that intersects the longitudinal axis 103 of the shaft 105 (as shown in FIG. 1D), the first helical thread 110 may include a first plurality of chevron shapes that are oriented toward (i.e., point toward) the distal end 102 of the shaft 105. Likewise, the second helical thread 120 may include a second plurality of chevron shapes that are oriented toward (i.e., point toward) the proximal end 101 of the shaft 105.

In some embodiments, the first plurality of chevron shapes and the second plurality of chevron shapes may be arranged in alternating succession along the shaft 105 of the fastener, (e.g., see FIG. 1D).

In some embodiments, a plurality of first interlocking spaces 161 and a plurality of second interlocking spaces 162 may be formed between the first helical thread 110 and the second helical thread 120 along the shaft 105 of the fastener.

In some embodiments, the plurality of first interlocking spaces 161 may be formed intermediate the first concave undercut surfaces 131 and the second concave undercut surfaces 132.

In some embodiments, the plurality of second interlocking spaces 162 may be formed intermediate the first convex undercut surfaces 141 and the second convex undercut surfaces 142.

In some embodiments, the plurality of first interlocking spaces 161 may be larger in size than the plurality of second interlocking spaces 162.

In some embodiments, the plurality of first interlocking spaces 161 and the plurality of second interlocking spaces 162 may be shaped and/or configured to interlock with bone/other tissues received therein to increase fixation of the fastener within the bone/other tissues and provide additional resistance against multi-axial forces that may be applied to the fastener and/or the bone/other tissues.

In some embodiments, the plurality of second undercut surfaces 112 and the plurality of sixth undercut surfaces 126 may be angled toward each other to trap bone, soft tissues, other tissues, bone augment material(s), etc., within the plurality of first interlocking spaces 161 in order to increase fixation and resistance against multi-axial forces.

In some embodiments, the plurality of third undercut surfaces 113 and the plurality of seventh undercut surfaces 127 may be angled toward each other to trap bone, soft tissues, other tissues, bone augment material(s), etc., within the plurality of second interlocking spaces 162 in order to increase fixation and resistance against multi-axial forces.

In some embodiments, the plurality of first undercut surfaces 111 and the plurality of fifth undercut surfaces 125 may each form an angle α with respect to the longitudinal axis 103 of the shaft 105, as shown in FIG. 1D.

In some embodiments, the angle α may be greater than 90 degrees.

In some embodiments, the plurality of second undercut surfaces 112 and the plurality of sixth undercut surfaces 126 may each form an angle β with respect to the longitudinal axis 103 of the shaft 105.

In some embodiments, the angle β may be less than 90 degrees.

In some embodiments, the plurality of third undercut surfaces 113 and the plurality of seventh undercut surfaces 127 may each form an angle θ with respect to the longitudinal axis 103 of the shaft 105.

In some embodiments, the angle θ may be approximately 90 degrees.

In some embodiments, the angle θ may be greater than 90 degrees.

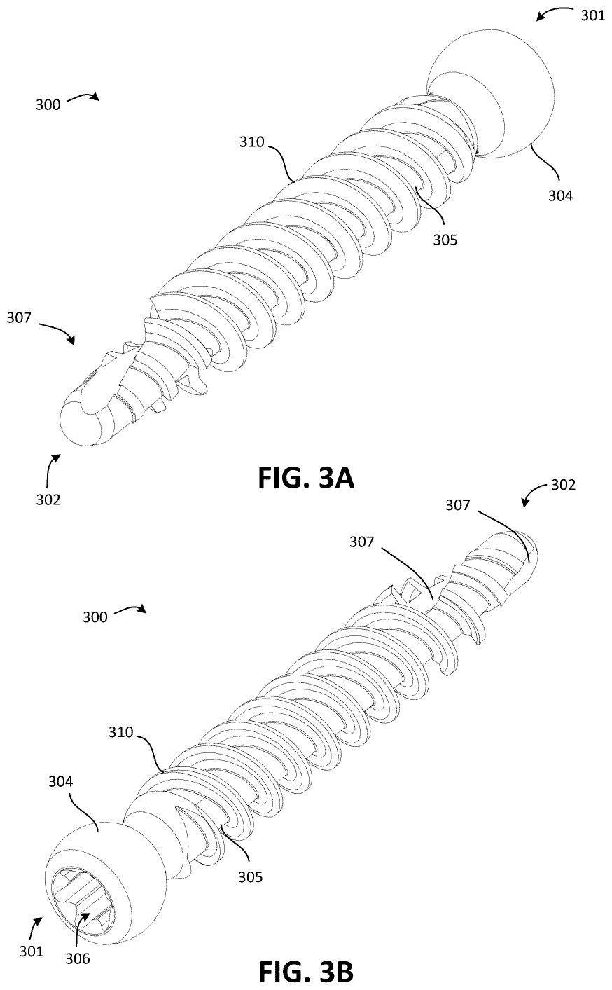

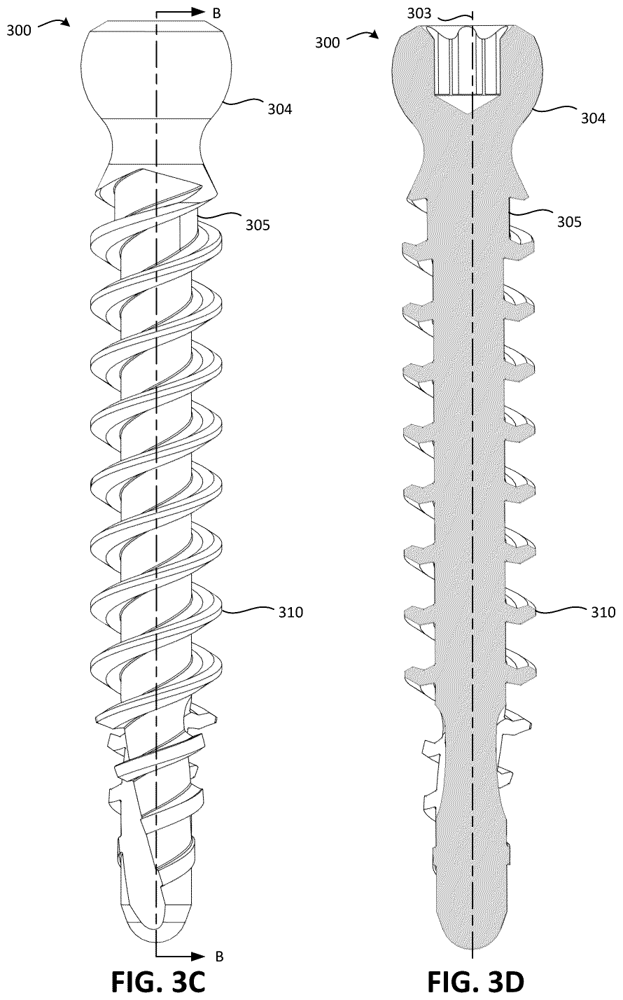

FIGS. 3A-3D illustrate various views of a fastener 300, according to another example of the present disclosure. Specifically, FIG. 3A shows a perspective distal end view of the fastener, FIG. 3B shows a perspective proximal end view of the fastener, FIG. 3C shows a side view of the fastener, and FIG. 3D shows a cross-sectional side view of the fastener taken along the line B-B in FIG. 3C.

The fastener 300 may similarly include a shaft 305 having a proximal end 301, a distal end 302, and a longitudinal axis 303. The fastener may also include a head 304 located at the proximal end 301 of the shaft 305, a torque connection interface 306 formed in/on the head 304, and a self-tapping feature 307 formed in the distal end 302 of the shaft 305.

In some embodiments, the fastener may include a helical thread 310 disposed about the shaft 305.

In some embodiments, the helical thread 310 may comprise “standard” threading having a “standard” orientation, as shown in FIG. 3D. However, it will be understood that the fastener may include any thread configuration, feature, or morphology described or contemplated herein to achieve optimal fixation within a given bone/tissue.

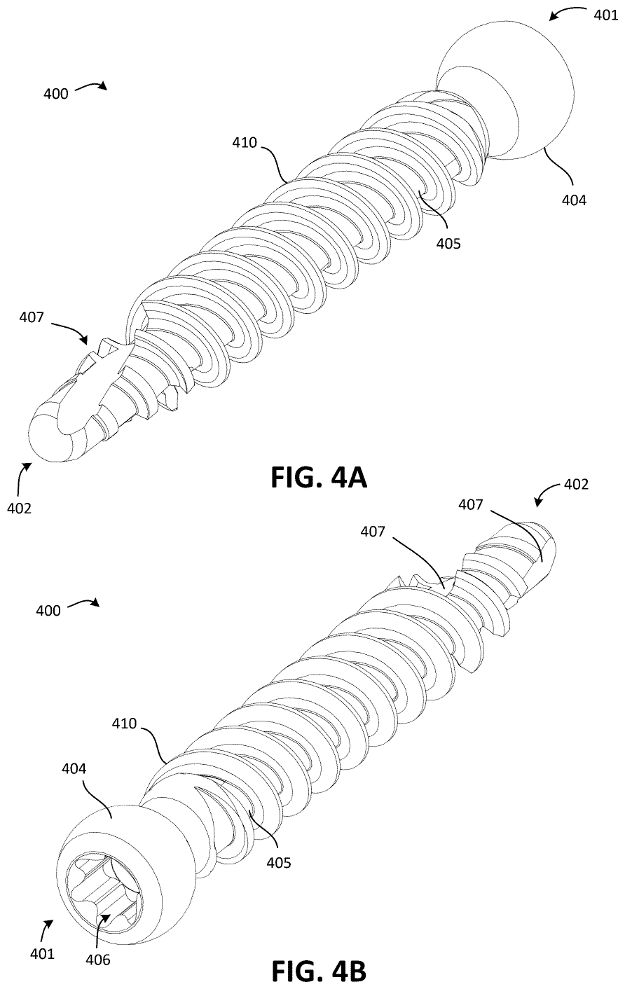

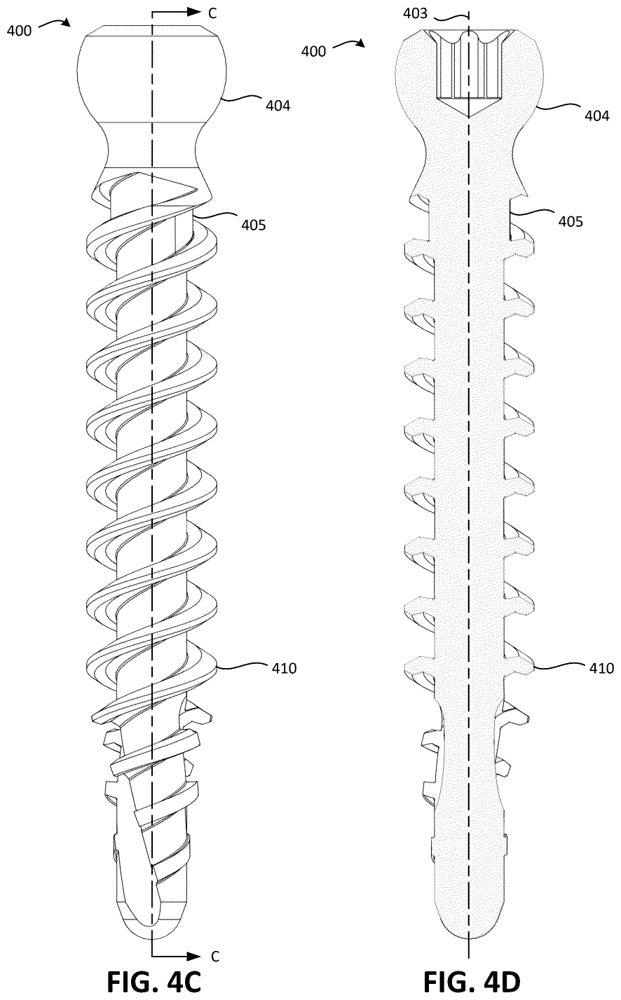

FIGS. 4A-4D illustrate various views of a fastener 400, according to another example of the present disclosure. Specifically, FIG. 4A shows a perspective distal end view of the fastener, FIG. 4B shows a perspective proximal end view of the fastener, FIG. 4C shows a side view of the fastener, and FIG. 4D shows a cross-sectional side view of the fastener taken along the line C-C in FIG. 4C.

The fastener 400 may likewise include a shaft 405 having a proximal end 401, a distal end 402, and a longitudinal axis 403. The fastener may also include a head 404 located at the proximal end 401 of the shaft 405, a torque connection interface 406 formed in/on the head 404, and a self-tapping feature 407 formed in the distal end 402 of the shaft 405.

In some embodiments, the fastener may include a helical thread 410 disposed about the shaft 405.

In some embodiments, the helical thread 410 may comprise “inverted” threading having an “inverted” orientation, as shown in FIG. 4D. However, it will be understood that the fastener may include any thread configuration, feature, or morphology described or contemplated herein to achieve optimal fixation within a given bone/tissue.

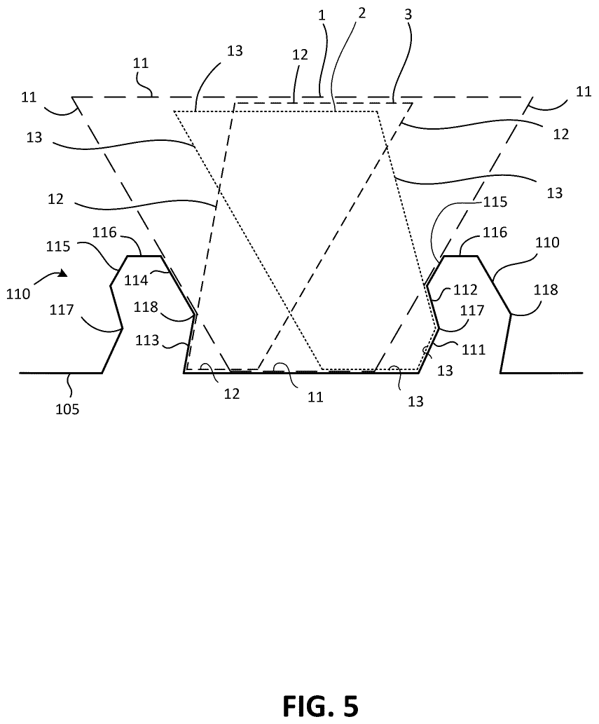

FIG. 5 shows a partial cross-sectional side view of a fastener undergoing various steps of a cutting process to form a first helical thread 110, according to an embodiment of the present disclosure.

In some embodiments, the first helical thread 110 of the fastener may be formed by a process that may include forming opposing undercut surfaces of the first helical thread 110 disposed about the shaft 105 of the fastener via a first cutting process, a second cutting process, and/or a third cutting process.

In some embodiments, the first cutting process may be performed with a first cutting head 1 of a first mill tool.

In some embodiments, the shape outlined by the four lines 11 shown in FIG. 5 may generally correspond to the shape of the first cutting head 1 for the first cutting process.

In some embodiments, the first cutting process may include: (1) placing the first cutting head 1 at a first position along the shaft 105 of the fastener; (2) rotating the shaft 105 of the fastener in a first rotational direction about the longitudinal axis 103 of the shaft 105 (e.g., if the first rotational direction is selected to be the “clockwise” rotational direction, then the corresponding opposite, second, or counter rotational direction would be in the “counter-clockwise” rotational direction, and vice versa); and (3) translating the first cutting head 1 along at least a first portion of a length of the shaft 105 to form the fourth open surface 114 and the fifth open surface 115 of the first helical thread 110.

In some embodiments, the second cutting process may be performed with a second cutting head 2 of a second mill tool.

In some embodiments, the shape outlined by the five lines 13 shown in FIG. 5 may generally correspond to the shape of the second cutting head 2 for the second cutting process.

In some embodiments, the second cutting process may include: (1) placing the second cutting head 2 at a second position along the shaft 105; (2) rotating the shaft 105 in the first rotational direction about the longitudinal axis 103 of the shaft 105; and (3) translating the second cutting head 2 along at least a second portion of the length of the shaft 105 to form the first undercut surface 111 and the second undercut surface 112 of the first helical thread 110.

In some embodiments, the third cutting process may be performed with a third cutting head 3 of a third mill tool.

In some embodiments, the shape outlined by the four lines 12 shown in FIG. 5 may generally correspond to the shape of the third cutting head 3 for the third cutting process.

In some embodiments, the third cutting process may include: (1) placing the third cutting head 3 at a third position along the shaft 105; (2) rotating the shaft 105 in the first rotational direction about the longitudinal axis 103 of the shaft; and (3) translating the third cutting head 3 along at least a third portion of the length of the shaft 105 to form the third undercut surface 113 of the first helical thread 110.

In some embodiments, the first cutting head 1, the second cutting head 2, and/or the third cutting head 3 may be rotated about the shaft 105 of the fastener during any of the above described cutting processes (e.g., via a whirling type thread forming process) in order to form at least a portion of the first helical thread 110 along the shaft 105 of the fastener (vs. rotating the shaft 105 rotating about its longitudinal axis 103 in a single-point type milling/lathing thread forming process). In these embodiments, the first cutting head 1, the second cutting head 2, and/or the third cutting head 3 may be rotated about a selected first rotational direction during each of these cutting processes, as previously described.

In some embodiments, two or more of: the first cutting process, the second cutting process, and/or the third cutting process may be performed simultaneously.

For example, in some embodiments at least two of: the first cutting head 1, the second cutting head 2, and/or the third cutting head 3 may each be simultaneously placed at different locations along the shaft 105 with respect to each other (e.g., at different locations along the same side of the shaft 105, on opposing sides of the shaft 105, etc.). In this manner, the cutting forces simultaneously applied to the shaft 105 from each of the cutting heads may act to cancel or balance each other out to help reduce movement/vibrations in the substrate or shaft 105 during the cutting process and achieve a more consistent/precise threadform.

In some embodiments, at least one cutting head may be placed on a first side of the shaft 105, and at least another one of the cutting heads may be placed on a second side of the shaft 105 at a selected degree of rotation about the longitudinal axis 103 of the shaft 105 relative to the first cutting head (or relative to any other cutting head that may be included). For example, the selected degree of rotation about the longitudinal axis 103 may comprise: two cutting heads separated from each other by 180 degrees of rotation about the longitudinal axis 103 of the shaft 105; three cutting heads separated from each other by 120 degrees of rotation about the longitudinal axis 103 of the shaft 105; four cutting heads separated from each other by 90 degrees of rotation about the longitudinal axis 103 of the shaft 105; five cutting heads separated from each other by 72 degrees of rotation about the longitudinal axis 103 of the shaft 105; six cutting heads separated from each other by 60 degrees of rotation about the longitudinal axis 103 of the shaft 105, etc. However, it will also be understood that any number of cutting heads may be placed about the shaft 105 at any degrees of rotation (e.g., with any equidistant degree of rotation, any non-equidistant degree of rotation, or any combinations thereof). Likewise, any number of cutting heads may be placed along the same side of the shaft 105 (i.e., zero degrees or rotation with respect to each other) according to any spacing/pattern (e.g., with any equidistant spacing/pattern, any non-equidistant spacing/pattern, or any combinations thereof).

In some embodiments, the first cutting process, the second cutting process, and/or the third cutting process may each be performed individually and/or in any sequence relative to each other (e.g., the third cutting process may be performed first or second, the second cutting process may be performed first or last, the first cutting process may be performed last or in between the other cutting processes, etc.).

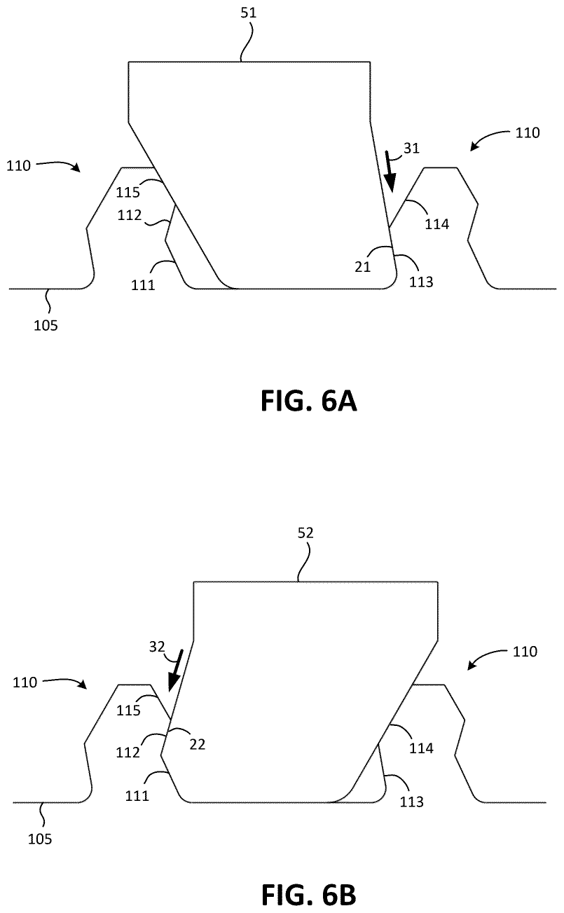

FIGS. 6A and 6B show partial cross-sectional side views of a fastener undergoing two steps of a cutting process to form a first helical thread 110, according to another embodiment of the present disclosure.

In some embodiments, the first helical thread 110 of the fastener may be formed by a process that may include forming opposing undercut surfaces on the first helical thread 110 via a first cutting process (e.g., see FIG. 6A) and a second cutting process (e.g., see FIG. 6B).

In some embodiments, the first cutting process may be performed with a first cutting head 51 of a first mill tool, and the second cutting process may be performed with a second cutting head 52 of a second mill tool.

In some embodiments, the first cutting process may include: (1) placing the first cutting head 51 at a first lateral position relative to the substrate or shaft 105 (e.g., above the substrate or shaft 105 prior to forming the first helical thread 110 thereon); (2) rotating the shaft 105 about a longitudinal axis 103 of the shaft 105 (alternatively, the first cutting head 51 may rotate about the shaft 105 via a whirling technique, etc., as previously described herein); (3) translating the first cutting head 51 medially toward the shaft 105 to at least one first medial position along a first oblique trajectory 31 defined by a first oblique cutting surface 21 of the first cutting head 51 (e.g., this step may be performed at increasing depths to slowly remove material from the substrate or shaft 105 and reduce friction, heat, etc., during the cutting process); and (4) translating the first cutting head 51 along at least a first portion of a length of the shaft 105 to form at least one of the opposing undercut surfaces on the first helical thread 110.

In some embodiments, the second cutting process may include: (1) placing the second cutting head 52 at a second lateral position relative to the shaft (e.g., above the substrate or shaft 105); (2) rotating the shaft 105 about the longitudinal axis 103 of the shaft 105 (alternatively, the second cutting head 52 may rotate about the shaft 105 via a whirling technique, etc.); (3) translating the second cutting head 52 medially toward the shaft 105 to at least one second medial position along a second oblique trajectory 32 defined by a second oblique cutting surface 22 of the second cutting head 52 (e.g., this step may be performed at increasing depths to slowly remove material from the substrate or shaft 105 and reduce friction, heat, etc., during the cutting process); and (4) translating the second cutting head 52 along at least a second portion of the length of the shaft 105 to form at least another one of the opposing undercut surfaces on the first helical thread 110.

In some embodiments, the first medial position may include a plurality of first medial positions along the first oblique trajectory 31 to achieve increasing cutting depths for the first cutting head 51, and the second medial position may include a plurality of second medial positions along the second oblique trajectory 32 to achieve increasing cutting depths for the second cutting head 52.

In some embodiments, the first cutting head 51 may be shaped to form the third undercut surface 113 and the fifth open surface 115 of the first helical thread 110, and the second cutting head 52 may be shaped to form the first undercut surface 111, the second undercut surface 112, and/or the fourth open surface of the first helical thread 110, as shown in FIGS. 6A and 6B.

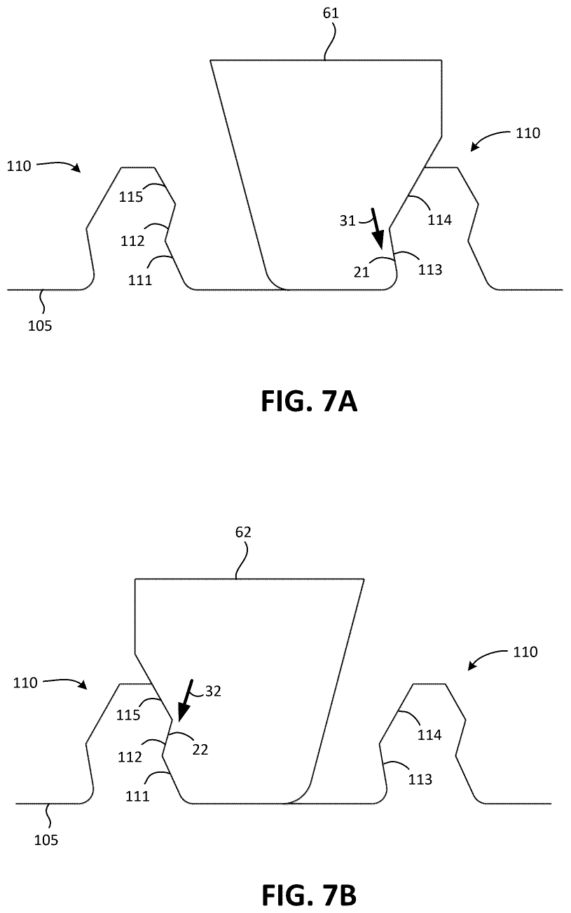

FIGS. 7A and 7B show partial cross-sectional side views of a fastener undergoing two steps of a cutting process to form a first helical thread 110, according to another embodiment of the present disclosure.

In some embodiments, the first helical thread 110 of the fastener may be formed by a process that may include forming opposing undercut surfaces on the first helical thread 110 via a first cutting process (e.g., see FIG. 7A) and a second cutting process (e.g., see FIG. 7B).

In some embodiments, the first cutting process may be performed with a first cutting head 61 of a first mill tool, and the second cutting process may be performed with a second cutting head 62 of a second mill tool.

In some embodiments, the first cutting process may include: (1) placing the first cutting head 61 at a first lateral position relative to the substrate or shaft 105 (e.g., above the substrate or shaft 105 prior to forming the first helical thread 110 thereon); (2) rotating the shaft 105 about a longitudinal axis 103 of the shaft 105 (alternatively, the first cutting head 61 may rotate about the shaft 105 via a whirling technique, etc.); (3) translating the first cutting head 61 medially toward the shaft 105 to at least one first medial position along the first oblique trajectory 31 defined by the first oblique cutting surface 21 of the first cutting head 61 (e.g., this step may be performed at increasing depths to slowly remove material from the substrate or shaft 105 and reduce friction, heat, etc., during the cutting process); and (4) translating the first cutting head 61 along at least a first portion of a length of the shaft 105 to form at least one of the opposing undercut surfaces on the first helical thread 110.

In some embodiments, the second cutting process may include: (1) placing the second cutting head 62 at a second lateral position relative to the shaft (e.g., above the substrate or shaft 105); (2) rotating the shaft 105 about the longitudinal axis 103 of the shaft 105 (alternatively, the second cutting head 62 may rotate about the shaft 105 via a whirling technique); (3) translating the second cutting head 62 medially toward the shaft 105 to at least one second medial position along the second oblique trajectory 32 defined by the second oblique cutting surface 22 of the second cutting head 62 (e.g., this step may be performed at increasing depths to slowly remove material from the substrate or shaft 105 and reduce friction, heat, etc., during the cutting process); and (4) translating the second cutting head 62 along at least a second portion of the length of the shaft 105 to form at least another one of the opposing undercut surfaces on the first helical thread 110.

In some embodiments, the first medial position may include a plurality of first medial positions along the first oblique trajectory 31 to achieve increasing cutting depths for the first cutting head 61, and the second medial position may include a plurality of second medial positions along the second oblique trajectory 32 to achieve increasing cutting depths for the second cutting head 62.

In some embodiments, the first cutting head 61 may be shaped to form the third undercut surface 113 and the fourth open surface 114 of the first helical thread 110, and the second cutting head 62 may be shaped to form the first undercut surface 111, the second undercut surface 112, and/or the fifth open surface 115 of the first helical thread 110, as shown in FIGS. 7A and 7B.

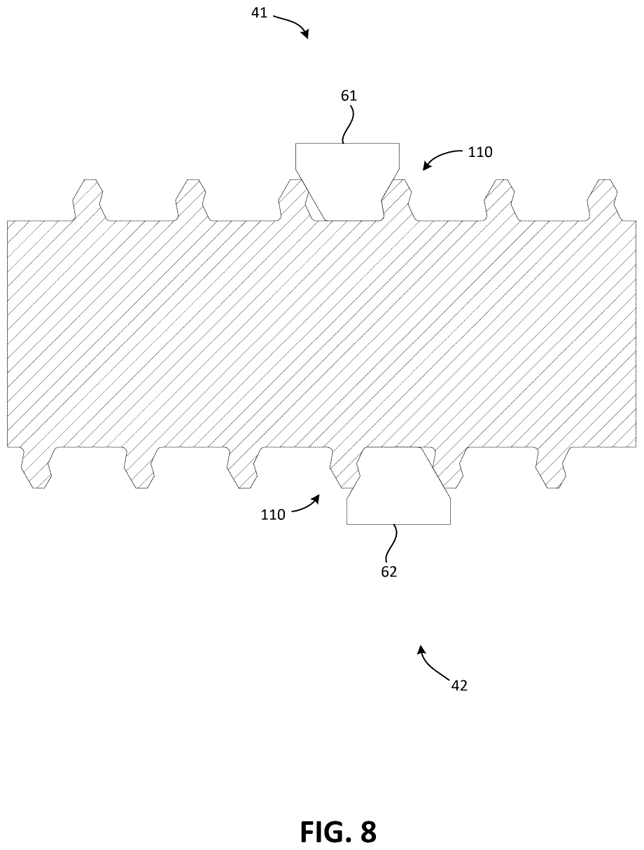

Referring to FIG. 8, the first cutting head (e.g., 61 or 51) may be placed on a first side 41 of the shaft at the first lateral position, and the second cutting head (e.g., 62 or 52) may be placed on a second side 42 of the shaft at the second lateral position. In this manner, both cutting processes may be carried out simultaneously to save time and/or “cancel/balance out” the opposing cutting forces applied to the shaft to help reduce movement/vibrations in the shaft during the cutting process and achieve a more consistent/precise threadform.

In some embodiments, a selected degree of rotation about the longitudinal axis of the shaft between the first cutting head (e.g., 61 or 51) and the second cutting head (e.g., 62 or 52) may be about 180 degrees, as shown in FIG. 8. However, any degree of separation may be utilized, as previously described herein. Moreover, the first cutting head (e.g., 61 or 51) and the second cutting head (e.g., 62 or 52) may be placed along the same side of the shaft (i.e., zero degrees or rotation with respect to each other) according to any spacing/pattern.

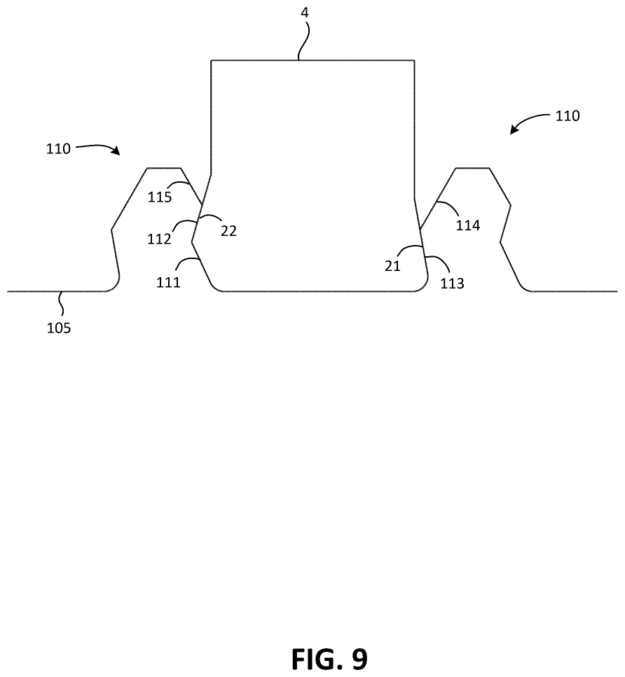

FIG. 9 shows a partial cross-sectional side view of a fastener undergoing a cutting process to simultaneously form at least two opposing undercut surfaces of a first helical thread 110, according to another embodiment of the present disclosure.

In some embodiments, the cutting process may be performed with a fourth cutting head 4 of a fourth mill tool.

In some embodiments, the cutting process may include: (1) placing the fourth cutting head 4 at a first position along the shaft 105; (2) rotating the shaft 105 about a longitudinal axis 103 of the shaft 105 (alternatively, the fourth cutting head 4 may rotate about the shaft 105 via a whirling technique, etc.); and (3) translating the fourth cutting head 4 along at least a portion of a length of the shaft 105 to simultaneously form the at least two opposing undercut surfaces on the first helical thread 110.

In some embodiments, the fourth cutting head may not rotate about a longitudinal axis of the mill tool during the cutting process that forms the at least two opposing undercut surfaces.

In some embodiments, the at least two opposing undercut surfaces may include the third undercut surface 113 on the first helical thread 110, and at least one of the first undercut surface 111 and the second undercut surface 112 on the first helical thread 110.

In some embodiments, the at least two opposing undercut surfaces may include the first undercut surface 111, the second undercut surface 112, and the third undercut surface 113 on the first helical thread 110.

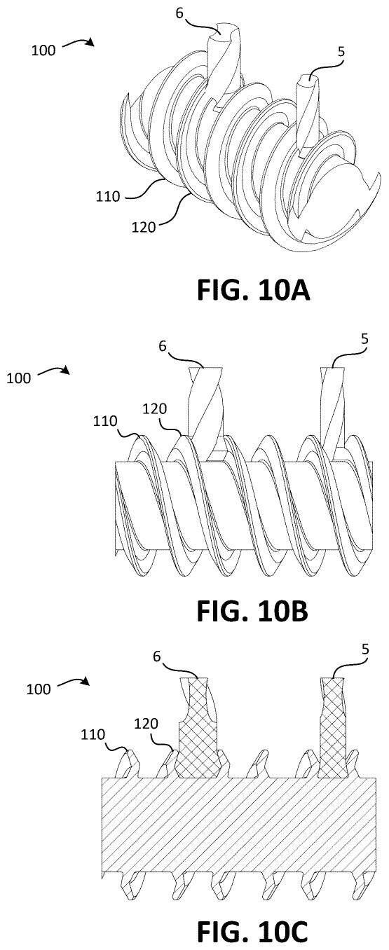

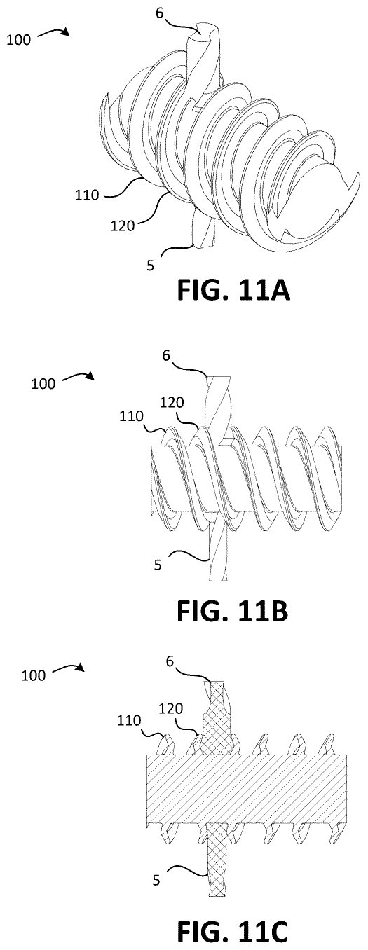

FIGS. 10A-11C show various views of a portion of the fastener 100 shown in FIGS. 1A-1D undergoing a cutting process to simultaneously form at least two opposing undercut surfaces of at least one helical thread of the fastener, according to other embodiments of the present disclosure. Specifically, FIG. 10A shows a perspective view of the fastener with the cutting heads on the same side of the fastener; FIG. 10B shows a side view of the fastener of FIG. 10A; FIG. 10C shows a cross-sectional side view of the fastener shown in FIG. 10B; FIG. 11A shows a perspective view of the fastener with the cutting heads on opposing sides of the fastener; FIG. 11B shows a side view of the fastener of FIG. 11A; and FIG. 11C shows a cross-sectional side view of the fastener shown in FIG. 11B.

In some embodiments, the cutting processes shown in FIGS. 10A-11C may be performed with a fifth cutting head 5 of a fifth mill tool, and/or a sixth cutting head 6 of a sixth mill tool.

In some embodiments, the cutting process may include: (1) placing the fifth cutting head 5 at a first position along the shaft 105 and/or placing the sixth cutting head 6 at a second position along the shaft 105 (e.g., on the same side, on opposing sides, with any degree of relative rotation, with any spacing/pattern, etc.); (2) rotating the shaft 105 about a longitudinal axis 103 of the shaft 105 (alternatively, the fifth cutting head 5 and/or the sixth cutting head 6 may rotate about the shaft 105 via a whirling technique, etc.); and (3) translating the fifth cutting head 5 and/or the sixth cutting head 6 along at least a portion of a length of the shaft 105 to simultaneously form the at least two opposing undercut surfaces on the at least one helical thread.

In some embodiments, the fifth cutting head 5 and/or the sixth cutting head 6 may not rotate about their longitudinal axes during the cutting process that forms the at least two opposing undercut surfaces.

In some embodiments, the at least one helical thread may include the first helical thread 110 disposed about the shaft 105 of the fastener, as well as the second helical thread 120 disposed about the shaft 105 of the fastener adjacent the first helical thread 110.

In some embodiments, the at least two opposing undercut surfaces may include the third undercut surface 113 on the first helical thread 110, and the seventh undercut surface 127 on the second helical thread 120, both of which may be simultaneously cut by the fifth cutting head 5.

In some embodiments, the at least two opposing undercut surfaces may include at least one of the first undercut surface 111 and the second undercut surface 112 on the first helical thread 110, and at least one of the fifth undercut surface 125 and the sixth undercut surface 126 on the second helical thread 120, both of which may be simultaneously cut by the sixth cutting head 6.

In some embodiments, the at least two opposing undercut surfaces may include the first undercut surface 111 and the second undercut surface 112 on the first helical thread 110, and the fifth undercut surface 125 and the sixth undercut surface 126 on the second helical thread 120, all of which may be simultaneously cut by the sixth cutting head 6.

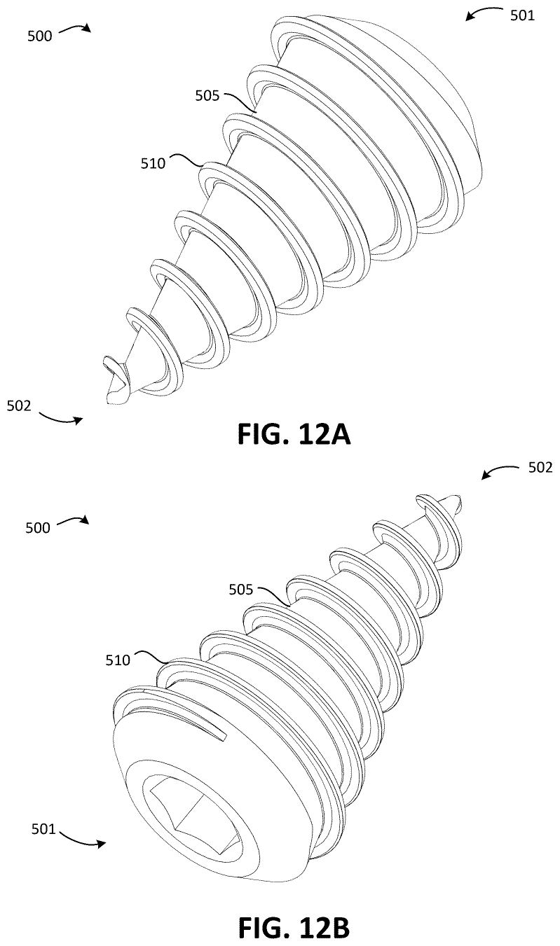

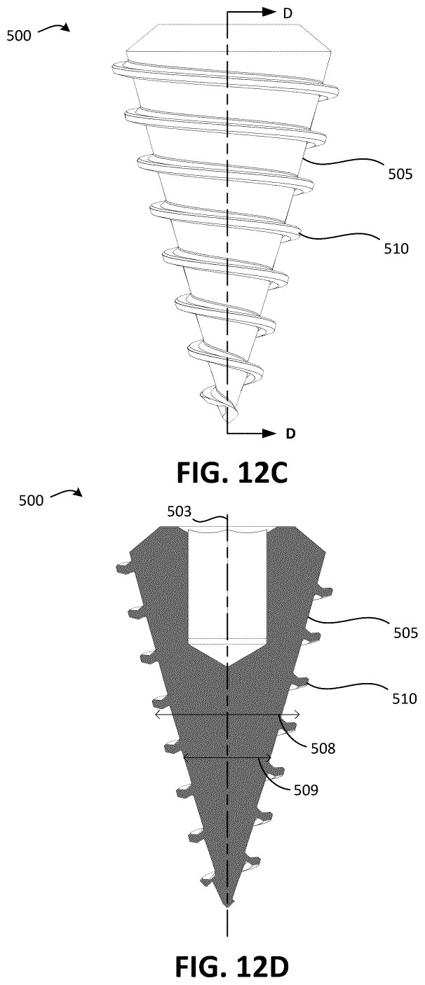

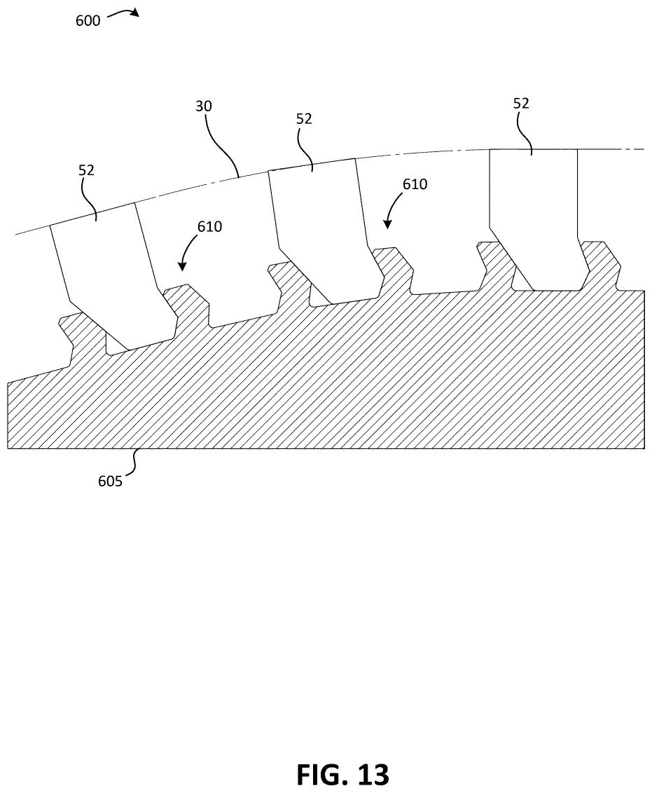

FIGS. 12A-13 illustrate various views of two example tapered fasteners, according to embodiments of the present disclosure. Specifically, FIG. 12A is a perspective distal end 502 view of a tapered fastener 500 comprising a conically tapered shaft 505 and a conically tapered helical thread 510; FIG. 12B is a perspective proximal end 501 view of the tapered fastener 500; FIG. 12C is a side view of the tapered fastener 500; FIG. 12D is a cross-sectional side view of the tapered fastener 500 taken along the line D-D in FIG. 12C; and FIG. 13 is a partial cross-sectional side view of a curved tapered fastener 600 comprising a curved tapered shaft 605 and a curved tapered helical thread 610.

These example tapered fasteners may (or may not) generally include similar features to other fasteners disclosed or contemplated herein, as previously discussed. However, the shafts and/or helical threads disposed about these example tapered fasteners may be shaped to taper in at least some manner.

In some embodiments, the shaft and/or helical threads of the example tapered fasteners disclosed or contemplated herein may have a variable minor diameter (e.g., see variable minor diameter 509 in FIG. 12D) generally defined by the shape of the taper corresponding to the shaft, and/or a variable major diameter (e.g., see variable major diameter 508 in FIG. 12D) generally defined by the shape of the taper corresponding to the helical thread disposed about the tapered shaft.

In some embodiments, the variable minor diameter and/or the variable major diameter may generally decrease moving from the proximal end of the shaft toward the distal end of the shaft. For example, the conically tapered shaft 505 of the tapered fastener 500 may generally decrease moving along the longitudinal axis 503 from the proximal end to the distal end of the conically tapered shaft 505.

In some embodiments, the variable minor diameter 509 and/or the variable major diameter 508 may comprise a conical shape or an at least partially conical shape, as shown in FIGS. 12A-12D.

In some embodiments, the variable minor diameter and/or the variable major diameter may comprise a curved tapered shape (e.g., see FIG. 13).

In some embodiments, the curved tapered shape may comprise a convex curved tapered shape, as shown in FIG. 13. However, in some embodiments the curved tapered shape may comprise a concave curved tapered shape (not shown).

In some embodiments, the curved tapered shape may comprise at least one non-curved tapered shape (e.g., see FIGS. 12A-12D).

In some embodiments, any of the curved tapered shapes disclosed or contemplated may transition to a straight or non-tapered shaft/thread portion via a radius or curved trajectory 30 (e.g., see FIG. 13). However, it will also be understood that any of the curved tapered shapes disclosed or contemplated herein may transition to a different taper angle, a different taper curvature, a different taper shape, etc.

In any event, helical threads disposed about any of the tapering shafts that are disclosed or contemplated herein may likewise be formed via any of the cutting processes disclosed or contemplated herein. This may be accomplished by adjusting the orientation of the cutting head during the cutting process to keep the cutting head normal, or substantially normal, to the specific shape of the tapered shaft as the cutting head translates along the tapering shaft (e.g., see FIG. 13). In this manner, a tapered helical thread may be formed along the tapered shaft of the fastener (e.g., see FIGS. 12D and 13).

Any procedures or methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112. It will be apparent to those having skill in the art that changes may be made to the details of the above-described embodiments without departing from the underlying principles set forth herein.

The phrases “connected to”, “coupled to”, “engaged with”, and “in communication with” may refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation (e.g., integrally formed as a single piece, etc.), components that are removably and/or non-removably coupled with each other, components that are functionally coupled to each other through one or more intermediary components, etc. The term “abutting” refers to items that may be in direct physical contact with each other, although the items may not necessarily be attached together. The phrase “fluid communication” refers to two or more features that are connected such that a fluid within one feature is able to pass into another feature. As defined herein the term “substantially” means within +/−20% of a target value, measurement, or desired characteristic.

While specific embodiments and applications of the present disclosure have been illustrated and described, it is to be understood that the scope of the present disclosure is not limited to the precise configurations and components disclosed herein. Various modifications, changes, and variations which will be apparent to those skilled in the art may be made in the arrangement, operation, and details of the devices, systems, and methods disclosed herein.

What is claimed is:

1. A method of manufacturing a fastener comprising:
forming opposing undercut surfaces of a helical thread disposed about a shaft of the fastener via a first cutting process and a second cutting process,
wherein:
the first cutting process comprises:
placing a first cutting head of a first mill tool at a first lateral position relative to the shaft;
rotating the shaft about a longitudinal axis of the shaft;
translating the first cutting head medially toward a first medial position relative to the shaft along a first oblique trajectory defined by a first oblique cutting surface of the first cutting head; and
translating the first cutting head along at least a first portion of a length of the shaft to form at least one of the opposing undercut surfaces on the helical thread; and
the second cutting process comprises:
placing a second cutting head of a second mill tool at a second lateral position relative to the shaft;
rotating the shaft about the longitudinal axis of the shaft;
translating the second cutting head medially toward a second medial position relative to the shaft along a second oblique trajectory defined by a second oblique cutting surface of the second cutting head; and
translating the second cutting head along at least a second portion of the length of the shaft to form at least another one of the opposing undercut surfaces on the helical thread.

2. The method of claim 1, wherein:
the first medial position comprises a plurality of first medial positions along the first oblique trajectory; and
the second medial position comprises a plurality of second medial positions along the second oblique trajectory.

3. The method of claim 1, wherein:
the first cutting head is shaped to form:
a third undercut surface of the helical thread; and
a fifth open surface of the helical thread; and
the second cutting head is shaped to form:
a first undercut surface of the helical thread;
a second undercut surface of the helical thread; and
a fourth open surface of the helical thread.

4. The method of claim 1, wherein:
the first cutting head is shaped to form:
a third undercut surface of the helical thread; and
a fourth open surface of the helical thread; and
the second cutting head is shaped to form:
a first undercut surface of the helical thread;
a second undercut surface of the helical thread; and
a fifth open surface of the helical thread.

5. The method of claim 1, wherein:
the first cutting head is placed on a first side of the shaft at the first lateral position; and
the second cutting head is placed on a second side of the shaft at the second lateral position;
wherein the first cutting head and the second cutting head are separated by a selected degree of rotation about the longitudinal axis of the shaft.

6. The method of claim 5, wherein the first cutting head and the second cutting head are separated by 180 degrees of rotation about the longitudinal axis of the shaft.

7. The method of claim 1, wherein:
the fastener comprises a tapered shape; and
the first cutting head and the second cutting head are translated along the tapered shape of the fastener to form the opposing undercut surfaces on the helical thread.

8. A method of manufacturing a fastener comprising:
forming at least two opposing undercut surfaces of at least one helical thread disposed about a shaft of the fastener via a cutting process by:
placing a cutting head of a mill tool at a first position along the shaft;
rotating the shaft about a longitudinal axis of the shaft; and
translating the cutting head along at least a portion of a length of the shaft to form the at least two opposing undercut surfaces on the at least one helical thread.

9. The method of claim 8, wherein the cutting head does not rotate about a longitudinal axis of the mill tool during the cutting process that forms the at least two opposing undercut surfaces.

10. The method of claim 8, wherein:
the fastener comprises a tapered shape; and
the cutting head is translated along the tapered shape of the fastener to form the at least two opposing undercut surfaces on the at least one helical thread.

11. The method of claim 8, wherein:
the at least one helical thread comprises a first helical thread disposed about the shaft of the fastener;
the cutting head is shaped to simultaneously form the at least two opposing undercut surfaces on the first helical thread as the cutting head is translated along the shaft; and
the at least two opposing undercut surfaces comprise:
a third undercut surface on the first helical thread; and
at least one of:
a first undercut surface on the first helical thread; and
a second undercut surface on the first helical thread.

12. The method of claim 11, wherein the at least two opposing undercut surfaces comprise:
the first undercut surface on the first helical thread;
the second undercut surface on the first helical thread; and
the third undercut surface on the first helical thread.

13. The method of claim 8, wherein:
the at least one helical thread comprises:
a first helical thread disposed about the shaft of the fastener; and
a second helical thread disposed about the shaft of the fastener adjacent the first helical thread;
the cutting head is shaped to simultaneously form the at least two opposing undercut surfaces as the cutting head is translated along the shaft; and
the at least two opposing undercut surfaces comprise:
a third undercut surface on the first helical thread; and
a seventh undercut surface on the second helical thread.

14. The method of claim 8, wherein:
the at least one helical thread comprises:
a first helical thread disposed about the shaft of the fastener; and
a second helical thread disposed about the shaft of the fastener adjacent the first helical thread;
the cutting head is shaped to simultaneously form the at least two opposing undercut surfaces as the cutting head is translated along the shaft; and
the at least two opposing undercut surfaces comprise:
at least one of:
a first undercut surface on the first helical thread; and
a second undercut surface on the first helical thread; and
at least one of:
a fifth undercut surface on the second helical thread; and
a sixth undercut surface on the second helical thread.

15. The method of claim 14, wherein the at least two opposing undercut surfaces comprise:
the first undercut surface on the first helical thread;
the second undercut surface on the first helical thread;
the fifth undercut surface on the second helical thread; and
the sixth undercut surface on the second helical thread.

16. A method of manufacturing a fastener comprising:
forming opposing undercut surfaces of a helical thread disposed about a shaft of the fastener via a first cutting process, a second cutting process, and a third cutting process,
wherein:
the first cutting process comprises:
placing a first cutting head of a first mill tool at a first position along the shaft;
rotating the shaft in a first rotational direction about a longitudinal axis of the shaft; and
translating the first cutting head along at least a first portion of a length of the shaft to form a fourth open surface and a fifth open surface of the helical thread;
the second cutting process comprises:
placing a second cutting head of a second mill tool at a second position along the shaft;
rotating the shaft in the first rotational direction about the longitudinal axis of the shaft; and
translating the second cutting head along at least a second portion of the length of the shaft to form a first undercut surface and a second undercut surface of the helical thread; and
the third cutting process comprises:
placing a third cutting head of a third mill tool at a third position along the shaft;
rotating the shaft in the first rotational direction about the longitudinal axis of the shaft; and
translating the third cutting head along at least a third portion of the length of the shaft to form a third undercut surface of the helical thread.

17. The method of claim 16, wherein two or more of:
the first cutting process;
the second cutting process; and
and the third cutting process are performed simultaneously.

18. The method of claim 17, wherein at least two of:
the first cutting head;
the second cutting head; and
and the third cutting head are placed on opposing sides of the shaft with respect to each other to balance two or more cutting forces applied to the shaft.

19. The method of claim 16, wherein:
the first cutting process;
the second cutting process;
and the third cutting process are performed individually in any sequence.

20. The method of claim 16, wherein the fastener comprises a tapered shape.
